(19) United States
(12) Patent Application Publication
(10) Pub. No.: US 2004/0117126 A1
(43) Pub. Date: Jun. 17, 2004

(54) Method of assessing and managing risks associated with a pharmaceutical product

(76) Inventors: Jeffrey E. Fetterman, Newark, DE (US); Gary H. Slatko, Greenville, DE (US); James K. Laird, West Chester, PA (US)

Correspondence Address: Pepper Hamilton LLP, One Mellon Center, 5oth Floor, 500 Grant Street, Pitttsburgh, PA, 15219, US

(21) Appl. No.: 10/722,262

(22) Filed: Nov. 25, 2003

Related U.S. Application Data
(60) Provisional application No. 60/428,981, filed on Nov. 25, 2002.
(60) Provisional application No. 60/467,827, filed on May 1, 2003.

(51) Int. Cl. G06F 17/60; G06F 19/00; G01N 33/48; G01N 33/50

(52) U.S. Cl. 702/19; 705/3

(57) ABSTRACT

A method for identifying and assessing risks associated with a pharmaceutical product. The method comprises identifying adverse events caused by a pharmaceutical product, identifying the failure modes in the process of using said pharmaceutical product that exposes patients to said adverse events, quantifying the potential effects of said failure modes to conduct a hazard assessment for purposes of evaluating the need to mitigate the failure mode, and designing a risk management program to manage the adverse events. In addition, the present invention includes a method for creating effective interventions for use in mitigating the risk of the pharmaceutical product. Such method includes the creation of educational materials which are continually evaluated and revised to achieve an expected level of effectiveness on a target audience.

### CROSS REFERENCES

This application relates to U.S. Provisional Application Serial Nos. 60/428,981, filed Nov. 25, 2002, and 60/467,827, filed May 1, 2003, the contents of which are incorporated herein by reference in their entirety.

### BACKGROUND

Many industries and organizations have implemented risk management strategies and programs to assess and manage risks associated with various systems, processes and products. Risk management programs have found wide application, particularly in high-risk industries such as manufacturing, environmental, food industries, and aviation.

Fault Tree Analysis, for example, was developed by Bell Telephone in 1961. Fault Tree Analysis analyzes a system and graphically identifies the potential events which may lead to an adverse event in the system. Rather than identify all potential adverse events, this methodology utilizes a weighted analysis to address only what are deemed to be the most undesirable of the adverse events. Once these adverse events are identified, the analysis turns to identifying and evaluating the potential failures in the system which may lead to those events.

Similarly, Root Cause Analysis was developed to uncover failures in processes which have led to adverse events. A Root Cause Analysis is effective at identifying failures at both the system and organizational levels and may be used to uncover common root causes that link a variety of errors. The process was designed to identify the root causes of adverse events which have already occurred, therefore, the Root Cause Analysis is not useful as for prospective analysis.

A widely adopted risk assessment processes is Failure Mode Effect Analysis (FMEA). The FMEA arose as an engineering technique which subsequently has been adopted by a variety of industries to prospectively define, identify, and eliminate or mitigate known or potential failures which may cause a hazard in the system under review. FMEA utilizes a systematic approach of identifying all potential failures in a system and then determining potential effects of each failure. The FMEA analysis ranks each potential failure based on its severity, expected frequency or occurrence, and detectability. A risk priority number is assigned to each failure which helps focus attention on developing potential interventions to mitigate such risks.

Probabilistic Risk Assessment is another risk management approach, which has been utilized in high risk environments. Probabilistic Risk Assessment prepares an analysis of the probability of occurrence of a particular consequence, the magnitude of that consequence, and an assessment of uncertainties related to that consequence. This assessment attempts to quantify the state of knowledge regarding a particular risk which can be used in strategic decision making. Logic diagrams are used to identify initiating events and other potential events which may ultimately lead to system failures. Event trees or event sequence diagrams are also used to develop the logical processes of intermediate events that occur and which may lead to the failed end state.

The National Aeronautics and Space Administration (NASA) has developed a multi-tiered analysis for identifying, understanding, and controlling the potential risks of a given activity. The NASA process has the goal of continually assessing potential risks, ranking such risks based on degree of hazard and importance, implementing strategies to manage those risks, and insuring the effectiveness of these strategies through continual communication and documentation.

Two additional governmental agencies that manage risks, the Environmental Protection Agency and the Federal Aviation Administration, have developed risk assessment strategies to identify potential hazards, analyze such hazards to identify the potential causes and effects of the hazards, and develop reporting systems which capture the effectiveness of the systems.

Closely related to the direct assessment of risks, a variety of quality control mechanisms have been developed and used to manage risks in order to reduce inappropriate variations in processes and ensure quality products and proven outcomes. For example, Six Sigma is a quality improvement method developed by Motorola, Inc. which seeks to reduce or prevent failures in a given system to a negligible level of 3.4 occurrences per million. Generally, Six Sigma focuses on defect prevention, cycle time reduction, and elimination of excessive costs by defining the goals of the improvement activity, measuring the existing system to establish a baseline measurement, analyzing the system to identify ways to eliminate gaps between current performance and ideal performance, improving the system to find innovative ways to implement efficiencies without undermining quality, and controlling the system by insuring accountability for use of new guidelines or methods.

The International Organization of Standards in Geneva, Switzerland, has developed the ISO9000 quality standards for purposes of implementation within an interconnected system of processes. The ISO9000 guidelines provide for the management of quality and resources while maintaining procedures to measure the effectiveness of these guidelines for evaluation of the improvement and identification of additional improvement opportunities.

Recently, the health care industry has recognized that there is a need to implement risk management procedures. Procedures to increase patient safety and minimize potential hazards have received increased attention and scrutiny at both the regulatory level and in the popular media. By one estimate, more than one million people in the United States suffer from preventable medical injuries with as many as 100,000 deaths annually resulting from those injuries. Medical injuries and deaths occurring at this rate would place medical errors as the eighth leading cause of death in the United States, ahead of both breast cancer and AIDS.

Following a series of highly publicized, fatal medical errors at Veterans' Affairs (“VA”) hospitals, the VA adapted the Failure Mode and Effect Analysis for use in hospital facilities as a means to implement patient safety initiatives. This effort resulted in the Healthcare Failure Mode and Effect Analysis (“HFMEA™”). The HFMEA™ methodology uses a team approach to diagram and identify failure modes and causes, to arrive at a hazard score and to utilize a decisional algorithm for identifying system vulnerabilities. The HFMEA™ process results in a prioritized schedule for failure modes and their causes, which is used to proactively allocate resources to address the particular risk factors.

The pharmaceutical industry has made limited attempts to manage the risks associated with a particular drug, in the form of risk communications and risk management programs. These attempts have often been reactive, developed on an ad hoc basis and have only had marginal success. Commonly, pharmaceutical manufacturers have utilized warnings in product information (e.g. black box warnings), and “dear health care provider” letters. These direct communications from the pharmaceutical company to the physician responsible for prescribing the drug, warn the physician of potential risk factors associated with the drug itself. These interventions have been utilized in conjunction with a variety of pharmaceuticals and have proven, by themselves, to be ineffective at adequately mitigating the risks associated with adverse side effects.

For example, Duract® (bromfenac) is a non-steroidal, anti-inflammatory drug introduced in 1997 for short-term pain relief. Although no known adverse effects were present when Duract® was introduced to the market, post marketing reports of severe liver damage among patients surfaced when Duract® was used for long-term pain management. The manufacturer placed black box warnings on the drug's label indicating that it should not be used in excess of ten days. Separately, letters were sent to physicians by the drug manufacturer advising of potential liver damage associated with the prolonged use of the drug. Post intervention studies showed that the effect of both the black box warning and the letter to physicians was minimal and, in June 1998, the drug was voluntarily withdrawn from the market.

Propulsid® (cisapride) was introduced to market in August 1993 for nocturnal heartburn. By 1995, approximately 5 million outpatient prescriptions for the drug had been issued. There were also reports of cardiac arrhythmias, torsade de pointes and prolonged electrocardiographic QT intervals, which resulted in four fatalities. To mitigate the risk of these adverse side effects, a black box warning was added to the label of the drug itself and a letter was sent by the manufacturer to the health care professionals responsible for prescribing the drug. Subsequent studies of the effectiveness of both of these interventions have revealed that they were not successful in eliminating prescriptions to patients with contraindicated conditions or medications. As a result of these failures, in July 2000, Propulsid® was removed from the U.S. market.

In 1985, Seldane® (terfenadine) was introduced to the market as a prescription antihistamine which did not cause drowsiness. At its peak, Seldane had an 80% market share of all allergy drugs. However, complications such as QT prolongation arose with the use of the drug in combination with other pharmaceuticals. Label warnings were strengthened to address contraindications, and letters were sent to health care professionals responsible for prescribing the drug. As was the case with Propulsid®, these interventions were insufficient to satisfy safety and regulatory concerns. Ultimately Seldane® was also withdrawn from the market.

Current risk management efforts for improving patient safety while taking pharmaceutical products utilize additional risk management methods. These programs impose additional requirements, procedures and restrictions on physician prescribing, pharmacist dispensing and patient use of the medication. For example, Accutane® (isotretinoin) was introduced to the market in 1992 for the treatment of severe acne. Animal studies showed the drug was teratogenic in humans and therefore not appropriate for women who were or might become pregnant during therapy. The drug was introduced to market with direct warnings to physicians through direct mailings, package inserts, and advertisements that the at-risk population should not be prescribed the drug. As a result of continued reports of birth defects associated with Accutane®, an aggressive program to reduce the risk of pregnancy among women taking Accutane® was implemented. The interventions were multi-faceted and sought to continually remind potential patients of the adverse effects of the drug. The initial program: (a) required female patients to sign consent forms, (b) printed patient warnings on the drug capsule's blister packaging and inserts, and (c) made available a variety of informational materials by the manufacturer. In addition, the drug manufacturer paid for contraceptive counseling and provided a toll free number to report adverse effects. The drug manufacturer tracked the effectiveness of initial interventions in order to evaluate the effectiveness and to determine if modifications or additions were necessary.

In one of the most widely publicized cases of the adverse effects of a pharmaceutical, Thalomid® (thalidomide) was introduced in the 1950s to treat insomnia and morning sickness and was commonly prescribed to pregnant women. Thalomid® resulted in more than 10,000 reported birth defects, including missing or abnormal limbs, spinal cord defects, and other physical abnormalities. The drug was banned in the early 1960s, but reintroduced in 1998 with FDA approval for use in treating painful skin conditions associated with leprosy, lupus, rheumatoid arthritis, scleroderma, leukemia, and an array of cancers. The drug was reintroduced with a risk management procedure in place to ensure that fetal exposure to Thalomid® does not occur. A three-prong approach directed at the prescribing physician, patient and dispensing pharmacist was put in place. Prescribing physicians must be registered with the drug manufacturer in order to prescribe Thalomid®. Patients receive comprehensive counseling on the precautions associated with Thalomid®, mandatory contraception counseling and pregnancy testing, and are required to sign an informed consent form. Finally, pharmacists may only dispense Thalomid® with a prescription dated within seven (7) days of presentation. In addition, pharmacists are required to collect and retain signed consent forms from the patient, complete a patient registration form, and enroll the patient in a patient registry. Thalomid® may only be dispensed in amounts limited to a 28-day supply with no refills. Multiple information sources are provided to the prescriber and the patient.

Tikosyn® (dofetilide) was introduced to the market in 2000 to treat abnormal heart rhythms, atrial fibrillation, and atrial flutter. Possible side effects of the drug included abnormal, potentially life threatening heart rhythms such as torsade de pointes. To prevent this, the drug was introduced with a novel risk management program which requires all healthcare providers involved in the patient's treatment to complete an educational program prior to the patient starting Tikosyn® therapy. This educational program is not limited to physicians, but also includes institutions participating in patient care. In addition, the patient is required to be hospitalized for three days prior to initiating treatment with the drug or increasing the dosage of the drug. Finally, the patient is only able to obtain Tikosyn® through a single national mail order pharmacy. Studies to determine the effectiveness of this risk management program have revealed conflicting results. However, there is clear evidence that the program has negatively impacted the appropriate use of the drug and the willingness of physicians to prescribe the drug for their patients. A subsequent change to the program allows retail pharmacies enrolled in the program to dispense Tikosyn®.

Attempts have been made to move beyond programs that focus on traditional communication or restrictions. A program supporting Coumadin® (warfarin) is an example of a different marketing approach that can be applied to manage risks in a way that goes beyond communications or restrictions. In 1998, Coumadin® was the most commonly prescribed oral anti-coagulant and the eleventh most prescribed drug in the United States. Just eight years earlier, the drug was severely underutilized, because Coumadin® has a complex dose-response relationship, multiple drug-drug interactions, and a very narrow therapeutic range in which it works safely, all making its safe and effective use challenging. In order to overcome these challenges, a program was developed to increase physician confidence in prescribing and managing patients taking this drug. A patient tracking system was developed and implemented to provide a computerized method of storing and retrieving the information of patients taking the drug. In addition, educational tools are provided to patients along with support materials to assist and maintain the involvement of the patient's physician and in quality assurance reporting and analysis. Furthermore, an incentive program was implemented to recognize clinics which have demonstrated excellence in improving the quality of care for patients taking this drug therapy. As a result, sales of Coumadin® have steadily grown and the drug remains on the market today.

### SUMMARY OF THE INVENTION

Embodiments of the present claimed method utilize a number of methods and tools to manage risks associated with the use of pharmaceutical products. They include some selected from a group consisting of systematic research, assessment, specification, design, development and implementation of programs, services, tools, enablers, applications, assessments, measurement instruments, systems and other interventions to manage risks associated with the use and management of a pharmaceutical product.

The present invention preferably provides embodiments relating to risk management of a pharmaceutical product that specifically address the hazards resulting from the failure of a medication use process that should otherwise protect patients from-risk. In addition to specifically addressing the hazards, it is preferable that the methodologies employed within the present invention are effective in minimizing the hazards, and furthermore that they minimize the burden of implementation of a risk management program.

As stated above, the designed interventions are preferably effective in reducing the degree of hazard. More specifically, the methodologies of the present invention provide an effective set of interventions designed to utilize engineered communications that effectively mitigate risk in a way traditional communications are unable to achieve. Particularly, a comprehensive pharmaceutical product risk assessment and management method is provided which addresses each possible cause of failure with more than one intervention that protects patient safety. Moreover, the more than one intervention is specifically designed to be redundant in order to anticipate the failure of the primary intervention, coordinated with other interventions, and inclusive of educational techniques and implementation forums that maximize their effectiveness.

This application describes example methods that include a method of assessing and managing risks associated with a pharmaceutical product. In one example method, a pharmaceutical risk assessment and management method may include identifying an adverse event caused by using a pharmaceutical product, identifying failure modes of the medication use process, quantifying the potential effect of said failure mode to create a pharmaceutical-specific hazard score, conducting an assessment of the failure modes, and designing and implementing a risk management program to manage the adverse events associated with the use of the pharmaceutical product.

The present invention addresses the fact that many failures in the delivery of healthcare are due to human error. In a preferred embodiment, a method is provided which includes a primary intervention that reduces the incidence of specific human failures. Additionally, a method may include one or more redundant backup interventions that decrease the occurrence of failure or mitigate the consequences of failures when they occur. Any number of risk intervention techniques may be used alone or in combination in the creation of the program. These risk intervention techniques are utilized to design a program of risk management which preferably coordinates care among diverse care providers (such as physicians, pharmacists, patients, caregivers, other health care providers and the like) to simultaneously mitigate the hazards resulting from a medication use process failure in such a way which is both implementable and acceptable to physicians, pharmacists, caregivers, and patients. A number of risk intervention techniques, educational forums, practitioner tools, and medical insights that enable easy adoption and implementation by physicians, pharmacists, and patients are utilized in the present invention.

In a preferred embodiment, a method is provided for the use of adult learning and disease management principles and techniques designed to assure that the interventions are maximally effective in changing physician, pharmacist, and patient behavior.

In a preferred embodiment, a method is provided for the use of techniques to research, document, modify and incorporate tools, techniques and insights already developed by practicing clinicians into the design and implementation of risk management programs, in order to maximize ease of use and adoption.

### BRIEF DESCRIPTION OF THE DRAWINGS

FIG. 1 is a pharmaceutical product risk assessment and management method.

FIG. 2 is a block diagram of a pharmaceutical product risk assessment and management method.

FIG. 3 is a diagram of a medication use process method.

FIG. 4 is a diagram of a pharmaceutical hazard scoring matrix.

FIG. 5 is a block diagram of a method of developing an educational kit.

FIG. 6 is a diagram of a method of developing an on-line educational website.

FIG. 7 is a diagram of a method of developing a professional support network.

FIG. 8 is a graphical representation of a method of designing pharmaceutical product risk interventions.

FIG. 9 is a diagram of a method of developing a controlled distribution model.

FIG. 10 illustrates strategic implications of various models of risk management on the impact of drug utilization.

### DETAILED DESCRIPTION OF THE INVENTION

Other industries have utilized a systematic approach to risk assessment and management. Hospitals have adopted certain aspects of a failure mode and effects analysis. However, the pharmaceutical industry has largely approached these problems on only an ad hoc basis. The resulting programs have been only marginally effective. The results of an ad hoc approach to risk management can be failure to protect patient safety, loss of market share, and/or eventual removal of the pharmaceutical product from the market entirely.

Alternatively, an evidence-based and systematic approach to assessing and managing the risks of a particular pharmaceutical product can not only effectively mitigate potential adverse side effects, but can also do so without creating unnecessary barriers to the prescribing and use of the pharmaceutical product itself. Therefore, what is needed is a continuous systematic approach to assessing, managing, and measuring the risks associated with a pharmaceutical product, which is effective at balancing the objectives of patient safety, market effectiveness, and regulatory approval.

To support the success of products in gaining regulatory approval and physician adoption, an evidence-based methodology was designed to be used in pharmaceutical risk assessment and management in order to improve pharmaceutical product safety. The present method provides a continual and systematic assessment and management of the risks associated with the use of a pharmaceutical product. The present method incorporates the rigorously tested and well accepted principles of the Failure Mode and Effect Analysis, which has been developed and widely applied in the engineering, aerospace, military and other fields, to the assessment and management of risks associated with pharmaceutical products. In addition, example methods of selecting, designing and developing risk management interventions which will be effective in minimizing the occurrence of and mitigate the effect of any potential adverse side effects which may be caused by a given pharmaceutical product are provided. Finally, methods used to identify, document and incorporate specific kits, practitioner tools, medical insights and implementation forums in order to increase end-user implementation and effectiveness of intervention programs are described herein.

A systematically developed risk management program can effectively support the appropriate utilization and prescribing of pharmaceutical products. In contrast, a historical review of previously utilized pharmaceutical risk management programs demonstrates that, at best, there is an indeterminate effect on the level of prescribing of the pharmaceutical product and most likely a detrimental impact. One of the primary lessons to be learned from the past attempts to manage risks associated with pharmaceutical products is that the unintended consequences of implementing an inappropriately burdensome risk management program (e.g underusage of effective medicines in patients who would benefit) is greater than the upside associated with a properly designed program applied to a pharmaceutical product.

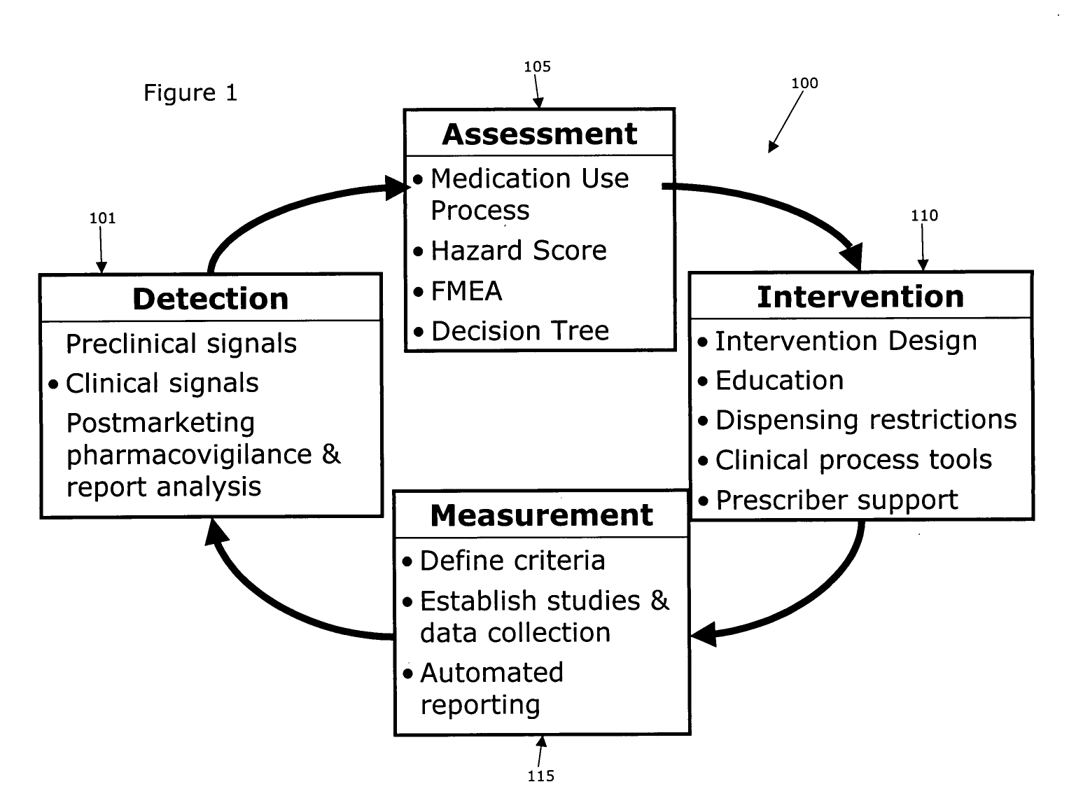

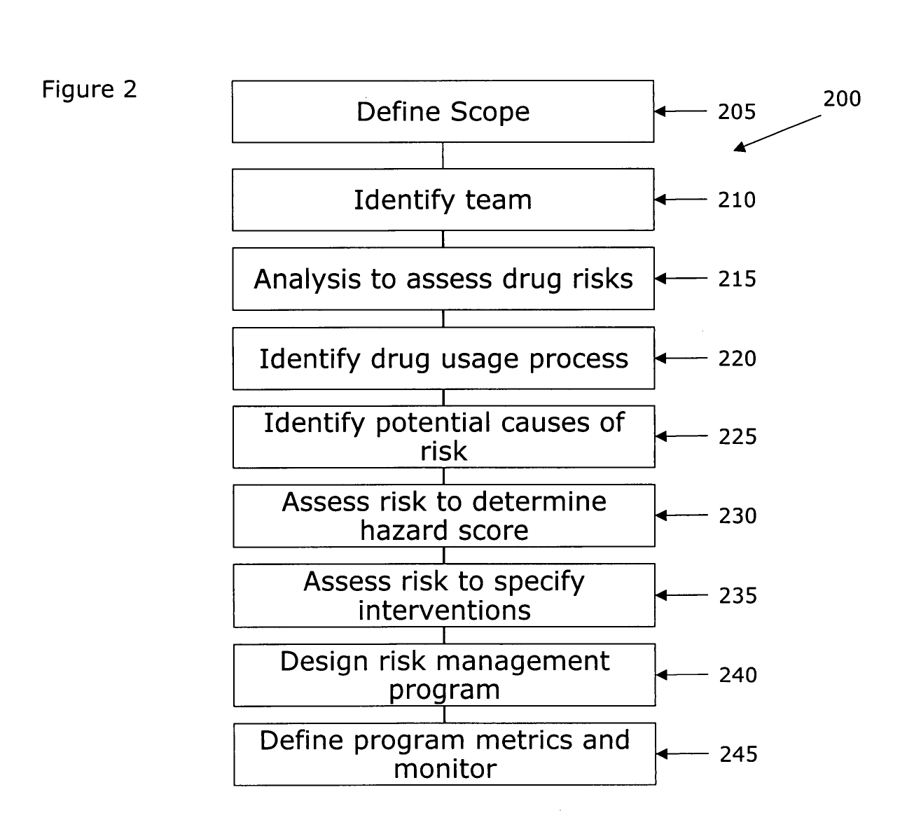

Aspects of the present invention may be better appreciated with reference to FIGS. 1 and 2. FIG. 1 illustrates an example method to assess and manage risks associated with a pharmaceutical product 100. The method utilizes a logical, evidenced-based process to develop a risk management program that at once will be less restrictive than an arbitrarily designed program, yet more likely to be accepted by regulatory authorities and implemented by end users. The method is a continuous process of Risk Detection 101, Risk Assessment 105, Risk Intervention 110, and Risk Monitoring and Measurement 115. By rigorously re-utilizing the framework shown in FIG. 1, the present method results in a well-adapted risk management program which continually evolves to meet the changing needs dictated by current and future hazards that may arise. Risk Detection 101 may include but is not limited to review of preclinical signals, clinical signal characterization and/or postmarketing pharmacovigilance and report analysis and the like to characterize signals of drug risk. Risk Assessment 105 may include but is not limited to the description of a medication use process and the utilization of a pharmaceutical hazard score, a Failure Mode and Effect Analysis (FMEA), and/or a decision tree and the like to evaluate where the process of medication use may fail to adequately protect patient safety. Risk Intervention 110 may include but is not limited to redundant and effective intervention design techniques, end user education, pharmaceutical product dispensing restrictions, clinical process tools, prescriber support systems, and/or implementation systems and forums and the like to reduce the incidence of process failure that may expose patients to drug risk. Risk Measurement 115 may include but is not limited to defining measurement criteria and systems, establishing studies, collecting data and/or automated reporting and the like to evaluate the effectiveness of the risk management program after implementation.

FIG. 2 is a block diagram of an example method of assessing and managing risks associated with a pharmaceutical product 200. As shown in FIG. 2, the method may include defining the scope of topic that will be evaluated by the process 205. The topic is customized for each risk management design project. Next, a multidisciplinary team of pharmaceutical experts may be assembled 210 to analyze and evaluate all of the data associated with the pharmaceutical product under review.

In the method 200, a variety of informational sources may be analyzed to detect the potential risks associated with the pharmaceutical product 215. This analysis 215 may encompass a review of preclinical and clinical data which may be generated in any regulatory approval process for the pharmaceutical product, safety data, pharmacogenomic information, literature associated with or related to the technology embodied in the pharmaceutical product, and customer interviews and the like. In addition, other risk management programs and tools in place both within the pharmaceutical industry and in other industries may be reviewed to determine whether this impacts the current analysis.

Once the potential risks associated with the pharmaceutical product are identified, an analysis of the medication use process may be conducted 220 by which it is intended that the pharmaceutical product reaches the end user. Identifying the medication use process 220 depicts and defines the sources from which the pharmaceutical product can be obtained, the method of dispensing and administering the pharmaceutical product, and the ability to monitor the safety and efficacy of the pharmaceutical product when taken by patients.

Once the medication use process 220 is identified, the method may include an analysis of the potential for failures of the existing process that could expose a patient to pharmaceutical product risk 225. Once the failure modes are identified 225, the possible effects of such failure may be assessed to determine a pharmaceutical hazard score 230. The pharmaceutical hazard score 230 is a quantification of the severity of the risk in relation to its frequency of occurrence according to pharmaceutical-specific severity and frequency scales.

Once a pharmaceutical hazard score for each failure has been determined 230, the risk may be assessed to determine whether interventions are needed 235, and, if so, a determination of which interventions may be appropriate to simultaneously control the risks while minimizing the negative affect on the use of the pharmaceutical product may be conducted. This risk assessment may be accomplished by use of a logical analysis.

Once the need for interventions has been identified 235, an appropriate risk management program may be designed and implemented 240. In designing the risk management program 240, an analytical view of the underlying potential causes of failure may be undertaken to determine the appropriate interventions. Selection of interventions may include consideration of the time burden required to perform the interventions, the operating costs associated with these interventions, and the potential impact the intervention will have on the target audience. In order to protect against the failure of primary interventions and to distribute program burden, redundant, multiple backup interventions may be created. Additionally, primary and redundant interventions are designed to be implemented by multiple healthcare providers. The design of risk management interventions may also employ adult learning techniques to enhance the effectiveness of individual interventions. Such techniques feature the integration of information, enabling tools, and personal action plans that enhance learning and behavior change. These techniques also guide the development of novel implementation forums and media that further enhance communication effectiveness. A measurement system and monitoring program may also be designed and implemented 245 to monitor the level of compliance with the various interventions and overall effectiveness of the program in reducing the risk, using predetermined metrics.

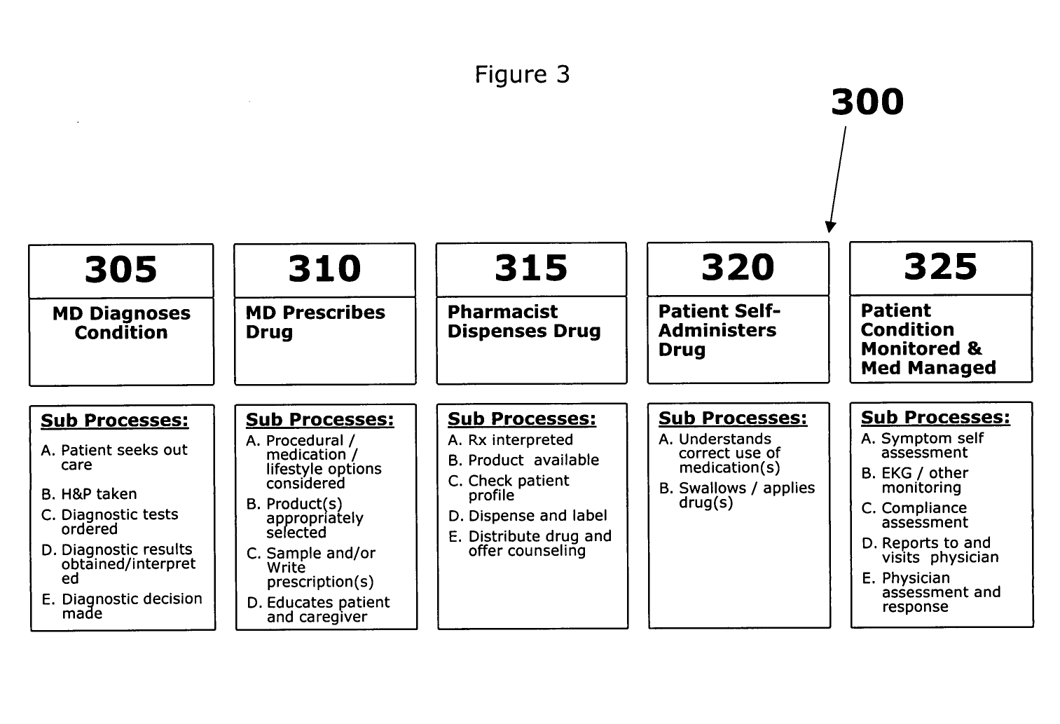

FIG. 3 sets forth a medication use process method 300 which may be utilized to identify the processes and subprocesses involved in providing a pharmaceutical product to an end user. As shown in FIG. 3, the method 300 may begin with a physician diagnosing a medical condition for which treatment may include use of a pharmaceutical product 305. Once the medical condition has been diagnosed 305, the method 300 may include a physician prescribing a pharmaceutical product 310. The method 300 may also include dispensing the pharmaceutical product by a licensed pharmacist 315. The method 300 may include self-administering the pharmaceutical product by the patient 320, and monitoring the patient's condition and further managing the pharmaceutical product 325. An important component of analyzing a medication use process in the present method is identifying the sub-processes associated with each of the general process steps in the overall medication use process. As shown further in FIG. 3, the sub-processes are identified for each step in the process and allow for the further detailed and refined analysis of the potential failure modes in the process that could expose a patient to adverse side effect risks associated with the product. The subprocesses of physician diagnosis 305 may include but are not limited to a patient seeking care, appropriate history and physical taken, appropriate diagnostic tests ordered, diagnostic results obtained and/or interpreted, diagnostic decision made and the like. The subprocesses of a physician prescribing a pharmaceutical product 310 may include but are not limited to considering procedural, medical and/or lifestyle options, selecting a pharmaceutical product, writing a prescription, educating the patient and/or caregiver and the like. The subprocesses of a pharmacist dispensing the pharmaceutical product 315 may include but are not limited to interpreting the prescription, assuring product availability, patient profile evaluation, dispensing and labeling, distributing the pharmaceutical product, offering opt-in counseling and the like. The subprocesses of a patient-self administering a pharmaceutical product 320 may include but are not limited to understanding correct medication use, swallowing the pharmaceutical product and the like. The subprocesses of monitoring a patient condition and managing the pharmaceutical product 325 may include but are not limited to symptom self assessment, lab monitoring, compliance assessment, reporting to and/or visiting the physician, physician assessment and response and the like.

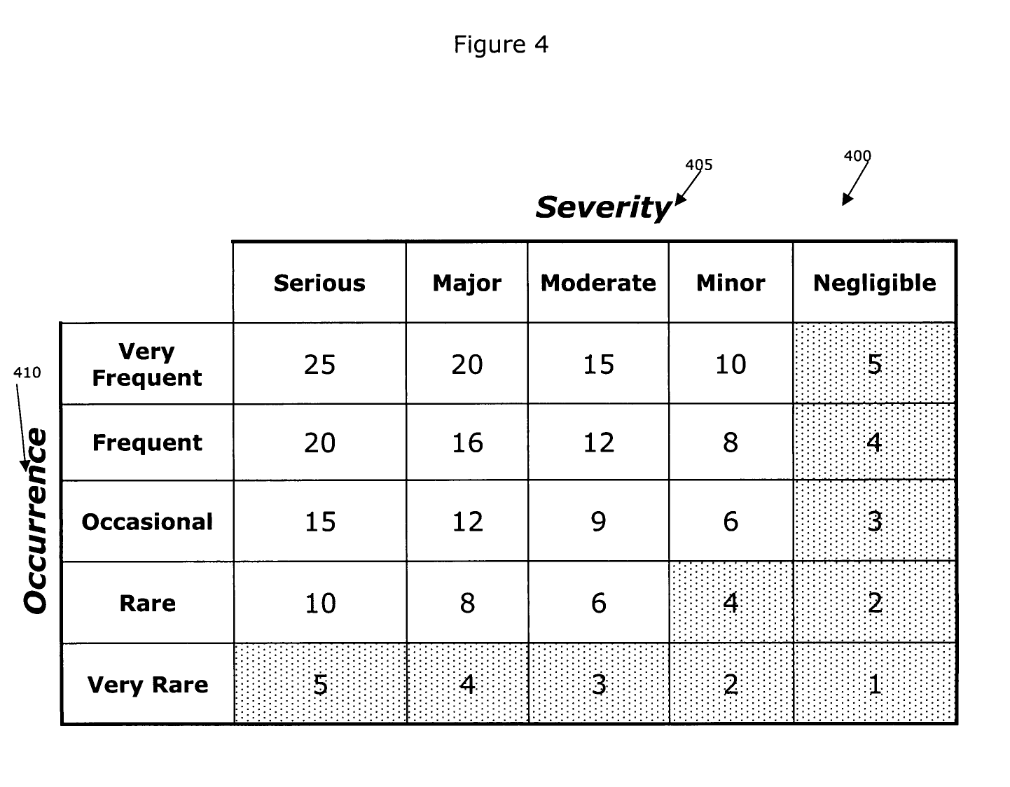

FIG. 4 illustrates an example pharmaceutical hazard score matrix 400 that may be utilized in a pharmaceutical product risk assessment and management method. Using the matrix in FIG. 4, the multi-disciplinary team can quantify the risk under review and better focus the analysis on those risks and failure modes which do not require action, those for which action may be required, and those for which action is definitively required. The possible effects of each failure mode on patient safety are assessed based upon the pharmaceutical hazard score. The pharmaceutical hazard score is calculated based upon pharmaceutical specific risk severity 405 and frequency of occurrence scales 410. An example pharmaceutical specific severity scale is illustrated in Table 1.

TABLE 1

| | | |
|---|---|---|
| 1 | Negligible | Slight HR or BP changes |
| 2 | Minor | Dizziness, transient low level lab abnormality, |
| | | mild QT prolongation up to 10 msec, BP |
| | | change of up to 5 mm Hg |
| 3 | Moderate | Fainting, orthostasis, transient moderate to |
| | | high level lab abnormality, moderate QT |
| | | prolongation of 10-20 msec |
| 4 | Major | Syncope, hospitalization, profound QT |
| | | prolongation of >20 msec, temporary |
| | | disability |
| 5 | Serious | Death, permanent disability, torsades de |
| | | pointes, malignant arrhythmia |

An example pharmaceutical specific frequency of occurrence scale is illustrated in Table 2.

TABLE 2

| | | |
|---|---|---|
| 1 | Very Rare | Less than 1/10,000 |
| 2 | Rare | From 1/10,000-1/1,000 |
| 3 | Occasional | From 1/1,000-1/100 |
| 4 | Frequent | From 1/100-1/10 |
| 5 | Very Frequent | Greater than 1/10 |

As shown in FIG. 4, the hazard score related to the effects of a specific failure increases as the severity of the risk increases and as the frequency of occurrence increases.

A multiple step logical analysis may be used in a pharmaceutical product risk assessment and management method to determine if a failure mode warrants intervention. Utilizing the pharmaceutical hazard score derived from the pharmaceutical hazard score matrix of FIG. 4, the interdisciplinary team determines whether the hazard involves sufficient likelihood of occurrence and severity to warrant further assessment. This is a flexible analysis which can be tailored to each pharmaceutical product, allowing appropriate risk management programs to be developed. For example, if more risk can be tolerated with the pharmaceutical product due to its nature or medication use process, the method permits establishing a higher threshold hazard score for taking action. If the hazard score indicates that control of the risk may not be necessary, the hazard is further evaluated to determine whether it is critical to the overall system, i.e, the occurrence of the hazard will likely result in a total system failure. If the hazard is not critical, then no additional risk management intervention is needed to manage this particular failure mode. If the hazard is critical, then an analysis of the existence of effective existing control mechanisms is conducted. If an effective control mechanism already exists, then no additional risk management intervention is needed to manage this failure mode. If an effective control mechanism does not exist, the analysis next focuses on whether the hazard is a detectable hazard. A detectable hazard is one which is so obvious and readily apparent that a control measure is not warranted. If the hazard is detectable, no additional risk management intervention is needed to manage this failure mode. If the hazard is not readily detectable, the risk assessment process proceeds into the design and implementation of effective interventions which are specifically tailored to manage and mitigate the risks associated with the pharmaceutical product.

If the hazard score indicates that control of the risk may be necessary, for example, the hazard score is greater than five, the analysis of the existence of an effective existing control mechanism is conducted. If an effective control mechanism already exists, then no additional risk management intervention is needed to manage this failure mode. If an effective control mechanism does not exist, the analysis next focuses on whether the hazard is a detectable hazard. A detectable hazard is one which is so obvious and readily apparent that a control measure is not warranted. If the hazard is detectable, no additional risk management intervention is needed to manage this failure mode. If the hazard is not readily detectable, the risk assessment process proceeds into the design and implementation of effective interventions which are specifically tailored to manage and mitigate the risks associated with the pharmaceutical product.

The design of effective pharmaceutical risk management interventions is a fluid process which must be adaptable to the specific needs of each hazard. Because of the recent emergence of the regulatory requirements for risk management, there are few sources of expertise which can be relied upon in designing risk management interventions. Further complicating the design of risk management interventions is that the goal of an intervention is to effect behavior changes by the physicians, pharmacists, healthcare providers and/or the patients who will use the pharmaceutical product. In the event that any of the parties chooses not to modify their behavior in relation to the pharmaceutical product, it is the manufacturer of that product who is held accountable, typically to regulatory authorities such as the U.S. Food and Drug Administration. The consequence of failing to modify the behavior is the occurrence of the hazard, which ultimately may result in withdrawal of the product from the market.

In an example method, a risk management program may include a primary intervention. The primary intervention for each failure mode should be customized to reduce the incidence of failure. For example, if the identified failure is that patients forget to adhere to the directions in administering the pharmaceutical product, an effective customized intervention would be frequent reminders to review and abide by the directions in administering the product. Examples of other methods of effective intervention may be found in Table 3.

TABLE 3

| | |
|---|---|
| Presentation Folder | Patient test schedule |
| Dear Healthcare Provider Letter | Patient diary |
| Overview Brochure | Patient ID wallet cards |
| Multiple Sample Forms | Patient Education Guidance for Practitioners |
| Observational Study Results Overview | Patient Education Booklet |
| Treatment Guidelines | Tablet dispenser |
| Dosing Chart | Audio tape |
| FAQ's | Video tape |
| Special Program Guidelines | Multilingual Patient Education |
| Reimbursement Guide Overview | Patient tracking binder/software |
| Patient Package Insert (PPI) | Issues and answers booklet |
| Study Outline/Data Sheet | Drug interaction chart |
| Patient Disease State Therapy Guide | Dietary Considerations |
| Caregiver Guide | Patient Newsletter |
| Medication ID Chart | Educational Assessment Form |
| Prescription Pads | Caregiver Guide |
| Conversion Tables | Prescription stickers-qualifications |
| Dosing & Administration Guide | Patient Agreement |
| Patient Information (English/Spanish) | Patient Informed Consent (male/female) |
| Steps for initiation of therapy | Schedule/appointment cards |
| Product Monograph | Medic Alert Information |
| On-line Education Program | Prescriber certification |
| Disease specific brochures | Guide to best practices |
| Outpatient initiation recommendations | Pregnancy Test Kit |
| Patient identification labels (colored) | Patient self-assessment |
| Flow sheets | Confidential patient counseling line (e.g. |
| | contraception) |
| Letter of understanding for prescribers | Continuing Medical Education |
| MD/RPh Prescribing procedures | |

In addition, the present method recognizes that the failure may occur despite the introduction of a primary intervention, therefore a risk management program may include redundant backup interventions, which are specified to the occurrences and mitigate the consequences of failures if they ultimately occur. Redundant interventions may include secondary level interventions, tertiary level interventions and the like. For example, a secondary intervention of educating office staff to provide patient counseling may be included to support or backup a primary intervention of educating physicians to provide patient counseling, in the event of failure of the primary intervention. In addition, an example method may include multiple communication vehicles used simultaneously to implement the interventions. It is known that learning can be affected in a variety of ways with varying degrees of effectiveness including reading, hearing, incorporating enabling tools, and developing a personal action plan to incorporate the learning into personal circumstances. In another example method, the use of the interventions may be temporally staged in order to appropriately match them to the period of greatest risk. In another example method, interventions may be designed with input from clinical end users to enhance their likelihood of implementation by other end users. In another example method, alternative venues for implementation, such as peer to peer interactive sessions, virtual learning labs, and preceptorships, and the like, may be incorporated.

In applying these principles to design an effective risk management program, due consideration is given to the ability to monitor and measure the program performance, its effectiveness, and implementability. This is an important feature which allows feedback and continuous program improvements. Furthermore, the design process seeks to avoid active interventions, such as disqualification, testing as a condition of drug access, controlled distribution, mandatory counseling and certification, and the like, because such active interventions tend to create barriers to the willingness of prescribing physicians and patients to utilize the product and realize its benefits. Such changes to the standard medication use process increase the probability of creating unintended consequences that would otherwise be avoided if the interventions were only educational or communicative in nature. In some cases, selected active interventions may need to be included as a contingency.

Common elements of a risk management program may include educational and counseling components and tools that are made available to the physician, the pharmacist, healthcare provider or the patient. The educational components include accredited or non-accredited continuing education programs for the physician, their office staff and the dispensing pharmacist. In addition, special labeling materials, which are to be physically placed on or within the drug containers themselves, may be included in an overall interventional program that is engineered to be effective. Educational and counseling components may include, for example, a Presentation Folder, Patient Lab Test Schedule, Dear Healthcare Provider Letter, Patient Diary, Overview Brochure, Patient ID Wallet Cards, Multiple Sample Forms, Patient Education Guidance for Practitioners, Observational Study Results Overview, Patient Education Booklet, Treatment Guidelines, Tablet Dispenser, Dosing Chart, FAQ's, Audio tape, Special Program Guidelines, Video tape, Reimbursement Guide Overview, Multilingual Patient Education, Patient Package Insert (PPI), Patient Tracking Binder/Software, Study Outline/Data Sheet, Issues and Answers Booklet, Patient Disease State Therapy Guide, Drug-Drug Interaction Chart, Caregiver Guide, Dietary Considerations, Medication ID Chart, Patient Newsletter, Prescription Pads, Patient Disease State Therapy Guide, Dose/Lab Conversion Table, Caregiver Guide, Dosing & Administration Guide, Prescription Stickers—Qualifications, Patient Information (English/Spanish), Patient Agreement, Steps for Initiation of Therapy, Patient Informed Consent (male/female), Product Monograph, Schedule/Appointment Cards, On-line Education Program, Medic Alert Information, Disease Specific Brochures, Prescriber Certification, Outpatient Initiation Recommendations, Guide to Best Practices, Patient ID Labels, Pregnancy Test Kit, Flow Sheets, Patient Self-assessment Flow Sheet, Confidential Patient Counseling Line, Letter of Understanding for Prescribers, Continuing Medical Education Credit, Physician Prescribing and Pharmacy Dispensing Procedures, Educational Assessment Form and the like.

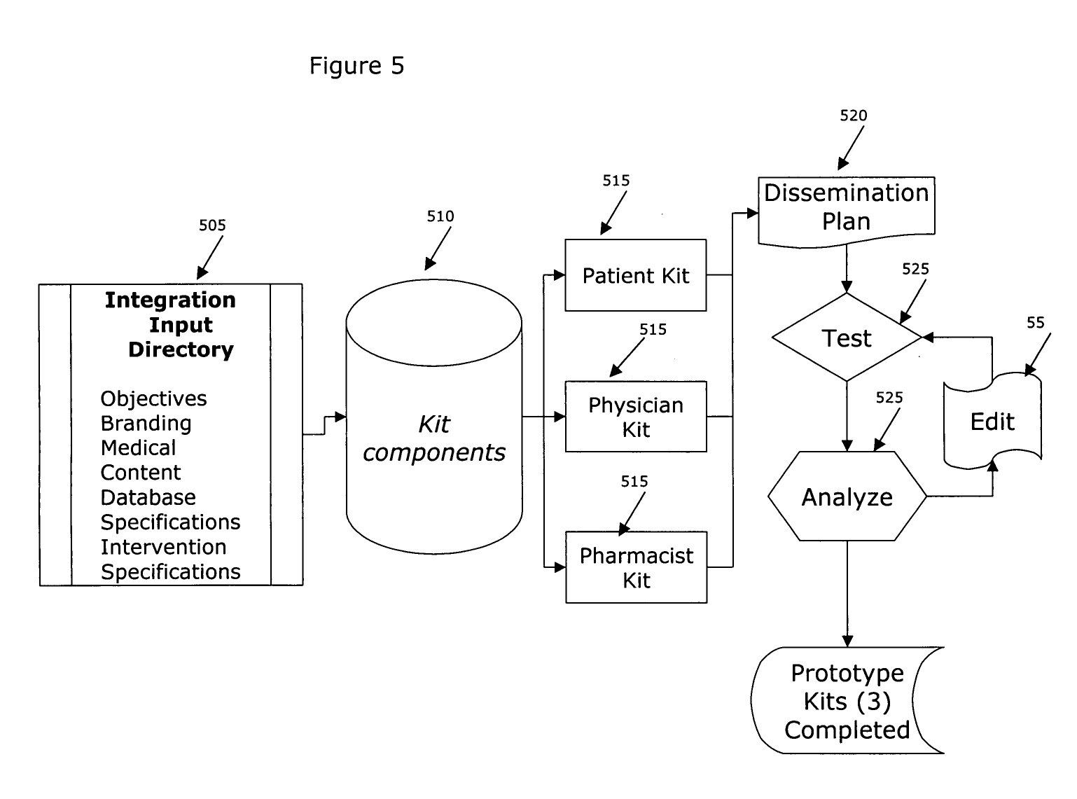

In another example of a risk management program element, a method for developing pharmaceutical product tool kits to be used as enablers of behavior change in managing the risk at the prescribing physician, pharmacist, and patient levels is provided, as illustrated in FIG. 5. Utilizing the objectives of the risk management program and the branding objectives of the company in conjunction with a medical content database 505, the key components are optimally selected from a database of existing educational components 510. For example, educational components 510 may include treatment guidelines, dosing charts, frequently asked questions, prescription pads, audio and videotapes describing the process of dispensing and using the pharmaceutical product, and a patient newsletter, and the like. The components 510 may include additional components such as those set forth in Table 3 and will be specifically tailored not only to the intended user of the toolkit itself, but also to the risk which is being managed. This tailoring is based on the inventors' Process of Care™ techniques and know how, whereby the practices, techniques, medical insights, and tools of clinical experts are researched, documented as a curriculum, and produced in the form of procedures, guides and tool kits. In this manner, the resulting tools are likely to be valued and easily implemented by other practitioners. The educational tool kits 515 may be prepared for a patient, physician, pharmacist, healthcare provider and the like.

In another example risk management program element, a dissemination plan 520 may be designed to disseminate the educational tool kits 515. The dissemination plan again will vary depending on the intended audience and may be implemented as part of other intervention programs. For example, if certification or counseling is a required element of the intervention program, the dissemination plan 520 may provide for distributing the educational kits 515 at that time. An important component of this design process is the continual testing and analysis of the effectiveness of the educational kit 515 at furthering the goals of the risk management program. Accordingly, a continual testing analysis and editorial loop 525 may be part of the process to ensure that the right elements are included in the educational kit 515. This test analysis and editorial loop 525 is designed to arrive at the optimal balance between having too many elements included in the kit which will tend to dilute the importance of the necessary items, and the failure to include enough of the items that should be included. Finally, a prototype kit for each of the patient, physician, and pharmacist will be completed and disseminated as per the plan.

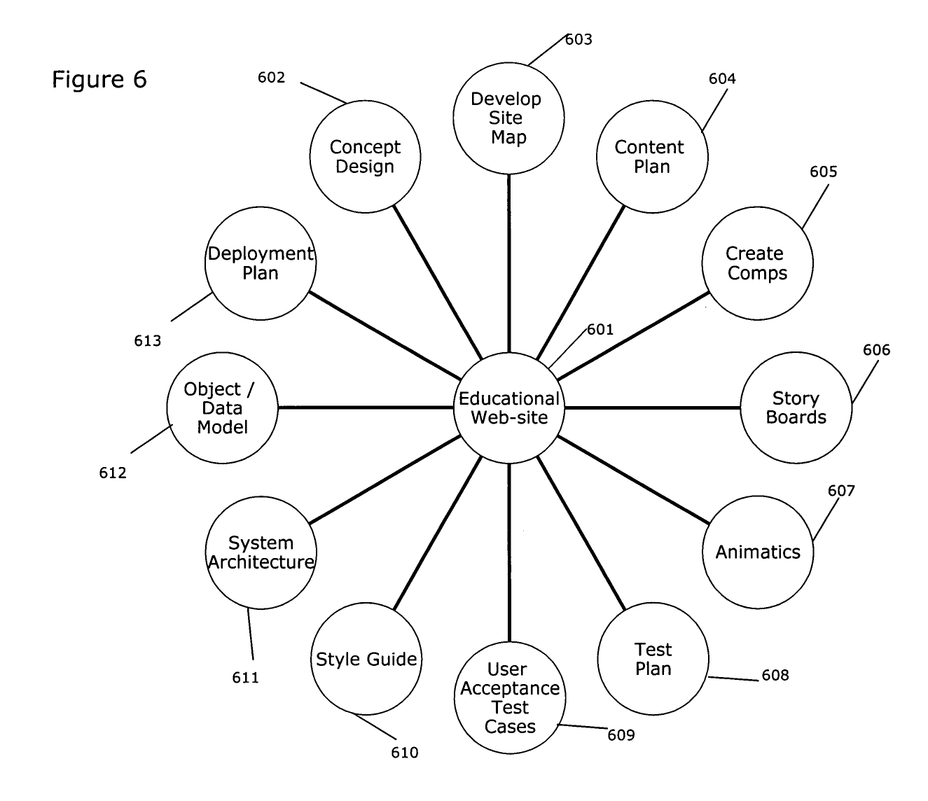

In another example risk management program element, an on-line education site may be created to deliver information to the patient as well as the physician and pharmacist. FIG. 6 illustrates an example on-line education site 601. The on-line education site 601 may be configured to implement the concept design and further the risk objectives identified in the analytical phase of the invention. The on-line education site 601 is highly flexible and may be tailored to the specific needs of the pharmaceutical product and its target audience. Various components may be used to adapt an on-line education site 601 to be specifically directed to the risks that have to be managed. Of these, one of the components of primary importance is the concept design 602. The concept design 602 will be determined by the number of risks to be managed, the diversity of those risks and the potential hazards posed by these risks. In addition to being an educational resource for patients, physicians, and pharmacists, the on-line site 601 will be effective at serving as a front-line filter for the manufacturer call center, thereby improving customer relations and ultimately the market presence of the pharmaceutical product itself. Other components used in creating and maintaining an on-line education 601 site may include but are not limited to Develop Site Map 603, Content Plan 604, Create Comps 605, Story Boards 606, Animations 607, Test Plan 608, User Acceptance Test Cases 609, Style Guide 610, System Architecture 611, Object/Data Model 612, Deployment Plan 613 and the like.

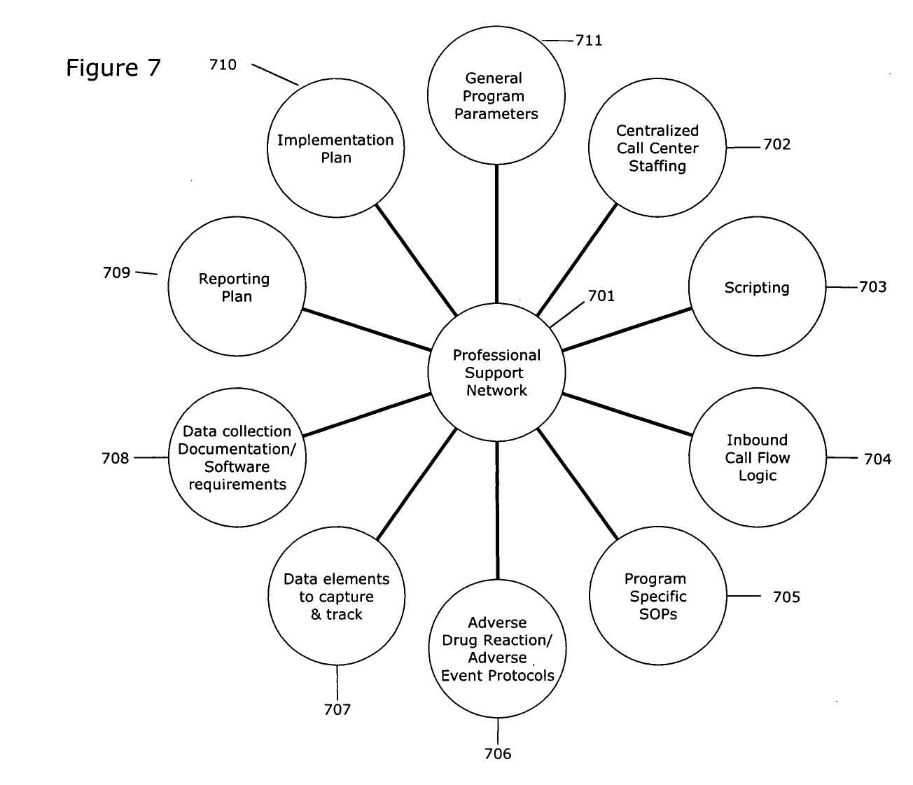

In another example risk management program element, a professional support network may be utilized as one of the methods of intervening to manage the risks associated with a pharmaceutical product. As shown in FIG. 7, the general program parameters of the support network 701 are to collect and manage data pertaining to clinical usage and established risk elements of the pharmaceutical product. In addition, the support network 701 would serve as a resource center for product support. Various components would comprise the typical professional support network 701 including a centralized call center 702 with scripted responses to expected questions 703, the process for receiving and responding to inbound calls regarding the pharmaceutical product 704, program specific standard operating procedures 705 and protocols 706 which will be utilized throughout the program generally and particularly in response to adverse events. An important component of the support network 701 will be data collection elements and either methods or software which capture, track 707, and document 708 the effectiveness of the network and its performance mitigating the risks associated with the pharmaceutical product. Additionally, a professional support network 701 may also include a reporting plan 709 and an implementation plan 710.

An example pharmaceutical product risk assessment and management method may include integration of the method into the overall marketing scheme for a pharmaceutical product. Given the flexibility of the present method in analyzing the risks associated with a particular pharmaceutical product and determining the appropriate intervention to create a risk management program, close coordination with the product marketing team to modify or adapt to the existing brand strategy and product profile to reflect the newly designed risk management program may increase the effectiveness of the method. For example, reviewing the tactics and messages to ensure that they are aligned with the requirements of the risk management program may help ensure that the effectiveness of the risk management interventions will be supported by the marketing effort and visa versa.

An example pharmaceutical product risk assessment and management method may include various methods of implementation and/or dissemination of the pharmaceutical risk assessment and management method. For example, methods of implementation and/or dissemination may include creation and utilization of end user education, 1-800 hotlines, physician protocols, end user support networks, and the like. The methods of implementation and dissemination may be directed to various end users, such as physicians, patients and healthcare providers. The methods of implementation and dissemination may involve collaboration among the end users, industry, and regulatory agencies. In another example, the method of implementation/and or dissemination may be designed to focus on various stakeholders, including the physician, patient and pharmacist, and adopt a variety of goals for each stakeholder, such as patient selection, prevention of co-prescribing and monitoring and managing side effects. Identifying and focusing on the stakeholders and goals enables the interventions to be designed and organized to more effectively assess and manage risk.

The facilitation and integration of the risk management program may be affected by documenting and submitting the program to the appropriate regulatory authority, such as the U.S. Food and Drug Administration, and considering any changes required by such regulatory authority in light of the particular medical requirements made necessary by the pharmaceutical product itself and in further light of the brand strategy which has been developed in conjunction with the management team. Revisions will be made considering these factors and resubmitted to the regulatory authority with the process being repeated as necessary.

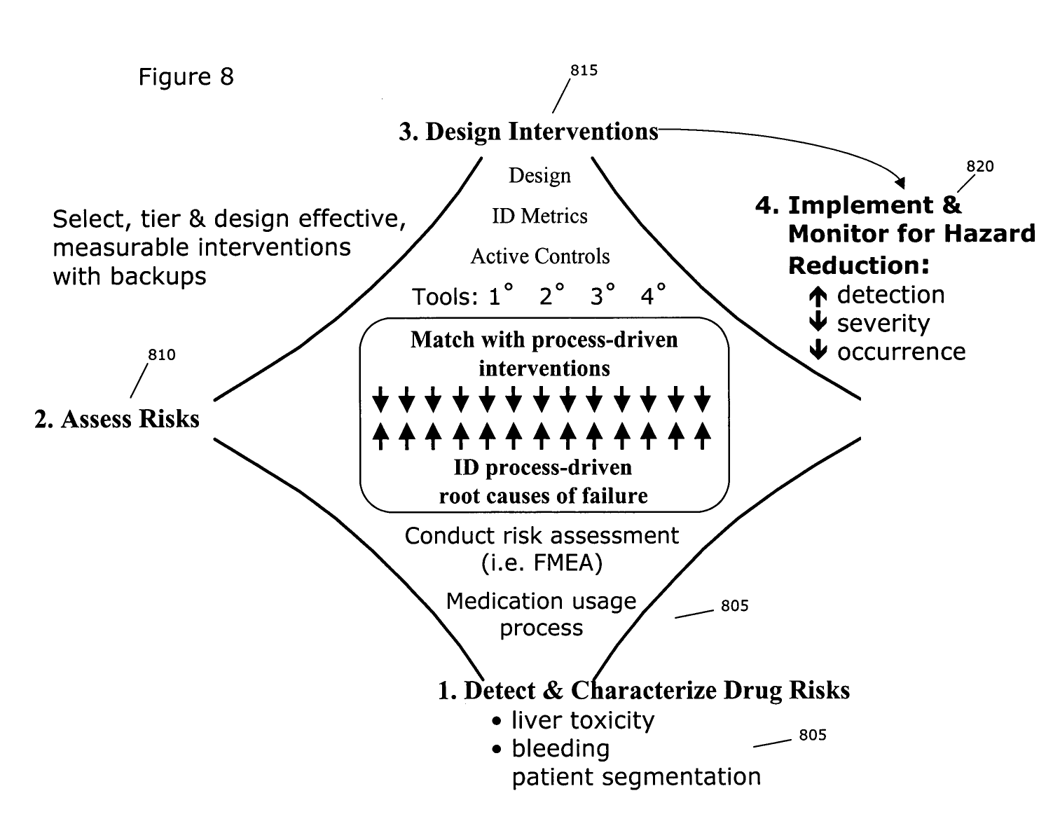

FIG. 8 is a diagram which graphically represents the development of risk interventions resulting from the risk assessment process. FIG. 8 shows the methodology employed in the present method for tailoring a risk management program utilizing known risk intervention procedures to reduce the incidence of failure that could expose patients to risks associated with adverse side effects of a pharmaceutical product. As shown in FIG. 8, intrinsic risks associated with the pharmaceutical product 805 are identified as is the medication use process for purposes of determining the severity of such risks. The medication use process is evaluated to identify weaknesses (failure modes) that allow patients to be exposed to the intrinsic risks of the drug 810. Additionally, a program of interventions is designed by assessing the severity and frequency of occurrence of each of the identified failure modes 815. Identification of potential interventions 815 is undertaken by specifying the type of communication or action that must be implemented to address each specific failure. Multiple redundant interventions are identified for each failure mode. The multiple interventions provide fail-safe backup and coordination of care among providers. Translating these specifications into useful tools is the role of the intervention designer who may visit medical experts in the field to observe the practices employed by experts when working with patients, may review published literature to evaluate the existence and effectiveness of various interventions for pharmaceutical products, or may evaluate the applicability of practices in other industries. Practices employed may include incorporating varying degrees of active controls based on the expected impact which the interventions may have. Such practices are documented, combined with similarly documented practices of other experts, and integrated into an intervention for use in the risk management program. Following this process, as shown in FIG. 8, risk interventions are specially designed for each of the failure modes to match the degree of active control necessary to mitigate the risk, if any 815. Outcomes metrics are identified for each intervention to allow monitoring of tool performance 820. This data is re-integrated and compared with the the initial hazard assessment to demonstrate the impact of the program on hazard scores.

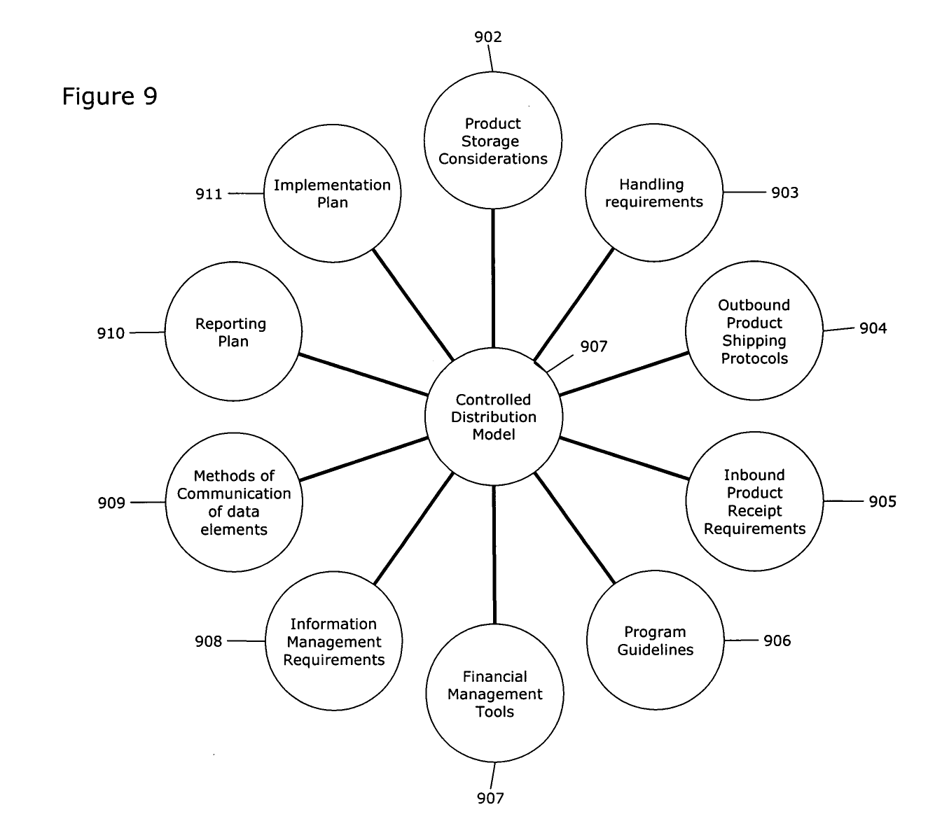

In the event that active control interventions are required to mitigate risk, certain controls may be added to the process of using medications including, for example, physician accreditation, pharmacist accreditation, physician attestation, pharmacist attestation, informed consent authorization, lab test result documentation, physician registry, patient registry, physician registration, pharmacist registration, physician certification, qualification stickers, sampling restrictions, special packaging, mandatory enrollment and follow-up survey, and the like. One example of an active control intervention, a controlled distribution model, may be utilized as shown in FIG. 9. A controlled distribution model 901 may be used to restrict access to a drug with safety issues if, for example, the risk communications alone are inadequate to mitigate the risk. The model shown in FIG. 9 is the basic parameter of a controlled distribution model 901 which may be adapted to a single source product distribution or to authorize distribution channels, as determined necessary from the risk management analysis performed by the present method. The process of developing a controlled distribution model 901 may entail numerous considerations such as product storage considerations 902, handling requirements for the drug itself 903, protocols for incoming and outgoing product 904 and 905 and the like. In addition, creation of program guidelines which advises all participants of the process of the parameters of the controlled distribution model is another component 906. Additional tools may be implemented to manage and monitor the financial and information components of the distribution model 907 and 908. As with other intervention procedures, an important component is tracking the data generated by the controlled distribution model and putting in place a reporting implementation plan 909, 910 and 911.

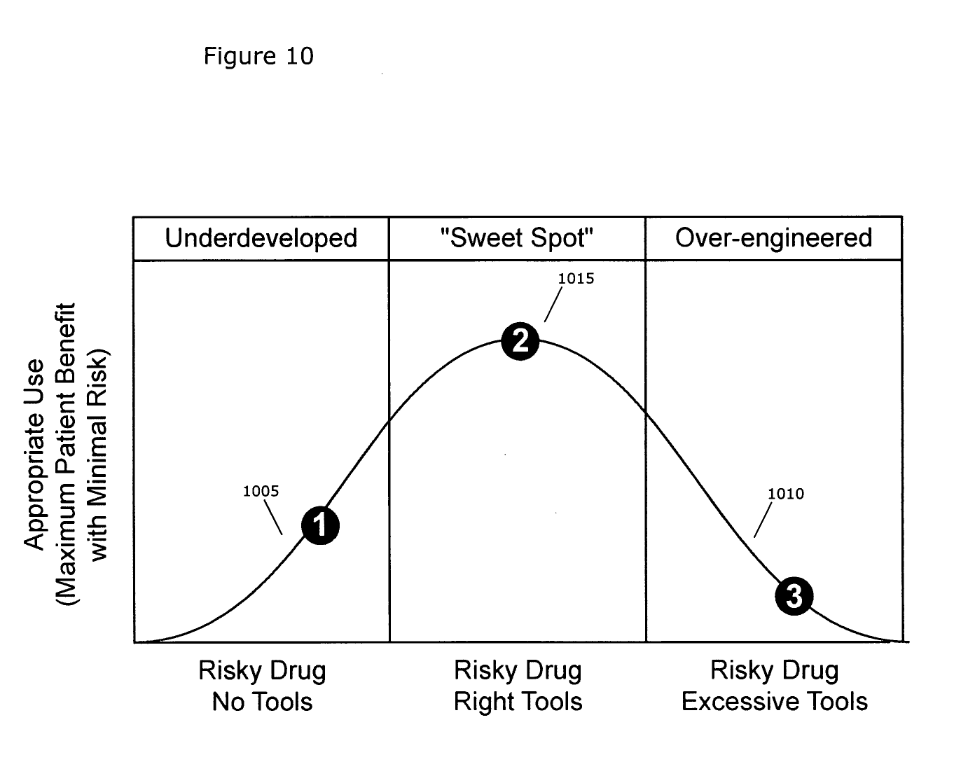

FIG. 10 illustrates the strategic implications of the various methods of pharmaceutical risk assessment and management on the impact of drug utilization. The present method improves patient safety, while taking into account that clinician perception dictates the actual ability to implement a risk management program. As shown in FIG. 10, clinicians will avoid using a pharmaceutical product if the risk management tools are deemed to be inadequate to reduce the hazard of the pharmaceutical product 1005. Physicians will also avoid using the product if the risk management program is deemed to be too burdensome on an end user 1010. Avoiding a pharmaceutical product may present a public health hazard because patients who may otherwise benefit from the pharmaceutical product do not have access to it. On the other hand, a well-designed program, as perceived by clinicians, enhances implementability, product adoption and potential clinical benefit 1015 for appropriate patients.

In the present method, risk detection analysis is used to identify intrinsic risks of the drug. Hazard assessment is used to identify which among those intrinsic risks are relevant to assess in the present methodology. The medication use process is evaluated to identify potential process-related failures in real world use that could expose patients to the intrinsic risks of drugs. Multiple redundant interventions are identified for each failure mode. The multiple interventions provide fail-safe backup, enable distribution and delegation of responsibility and foster coordination across care providers. If necessary, active controlling interventions may also be developed that actually change the medication use process if a particular failure cannot be addressed by tiered communications and education. An outcomes metric is identified for each intervention to allow monitoring of tool performance, while a measurement system is established to demonstrate performance against goals and objectives. This data is re-integrated into the hazard scoring algorithms to demonstrate the impact of the intervention on future pharmaceutical hazard scoring. Finally, the actual risk management interventions and tools are designed and developed using proprietary processes that engage physicians in the tool design in such a way that assures acceptability of the end program and enhances implementabality.

While the present invention has been described in conjunction with the exemplary embodiments outlined above, it is evident that many alternatives, modifications, and variations will be apparent to one ordinarily skilled in the art. Accordingly, the exemplary embodiments of this invention set forth above are intended to be illustrative, not limiting. Whereas modifications or changes may be made without departing from the spirit and scope of the invention, or may become obvious to one skilled in the art after review of the present invention, such modifications or changes are intended to be included within the scope of the present invention.

We claim:

1. A method for assessing and managing risks associated with utilizing a pharmaceutical product comprising:
identifying, characterizing and ranking adverse events caused by using the pharmaceutical product;
identifying a medication-use process associated with a pharmaceutical product;
identifying potential failure modes where the medication use process will not be adequate to protect patients from experiencing adverse side effects;
quantifying the potential effect of said failure mode to create a pharmaceutical hazard score, wherein said hazard score considers the severity and frequency of occurrence of the effects of said failure;
conducting a logical hazard assessment of said failure modes found to have a high hazard score to evaluate the need to mitigate the effect of said failure modes; and
designing a risk management intervention program to manage said adverse events.

2. The method of claim 1 further comprising:
implementing said risk management program.

3. The method of claim 2 further comprising:
measuring the effectiveness of said risk management program.

4. The method of claim 3 wherein measuring the effectiveness of said risk management program comprises:
measuring and defining metrics, measurement systems, program goals, objectives and program performance analysis and reporting.

5. The method of claim 3 further comprising;
integrating said effectiveness measurement into said pharmaceutical product hazard score.

6. The method of claim 5 wherein the step of integrating said effectiveness measurement comprises:
reporting said effectiveness measurement.

7. The method of claim 1 wherein the step of identifying, characterizing and ranking the adverse events caused by using the pharmaceutical product comprises:
analyzing available data from animal, toxicology, pharmacokinetic, pharmacodynamic and pharmacogenomic studies of the pharmaceutical product.

8. The method of claim 1 wherein the step of identifying, characterizing and ranking the adverse events caused by using the pharmaceutical product comprises:
analyzing existing clinical safety data for the pharmaceutical product.

9. The method of claim 1 wherein identifying, characterizing and ranking the adverse events caused by using the pharmaceutical product comprises:
analyzing risks identified in similar products.

10. The method of claim 1 wherein the step of identifying, characterizing and ranking said failure modes comprises:
graphically depicting the medication use process of prescribing, dispensing, and administering the pharmaceutical product as a plurality of steps; and
identifying subprocesses for each of said steps.

11. The method of claim 1 wherein identifying potential failure modes of the medication use process comprises:
identifying one or more processes of prescribing, dispensing or administering the pharmaceutical product or a combination thereof, and
optionally identifying subprocesses of said one or more processes.

12. The method of claim 1, wherein said pharmaceutical hazard score comprises:
utilizing a pharmaceutical severity scale; and
utilizing a pharmaceutical frequency of occurrence scale.

13. The method of claim 1 wherein the logical hazard assessment comprises:
analyzing the criticality and detectability of the failure mode to determine the need to mitigate the failure mode.

14. The method of claim 13 wherein the logical hazard assessment further comprises:
analyzing existing risk control measures for the failure mode to determine whether they are effective in mitigating the failure mode without further intervention.

15. The method of claim 1 wherein the risk management intervention program comprises:
effective education, communications and/or control measures in redundant combinations and incorporating adult learning principles designed to be readily implemented in order to effectively reduce the incidence and consequences of said failure modes.

16. The method of claim 1 wherein the risk management intervention program comprises:
a primary intervention targeted to reduce the incidence of each failure mode.

17. The method of claim 1 wherein the risk management intervention program comprises:
one or more redundant backup interventions to decrease the occurrence of and/or mitigate the consequences of failure of the primary intervention.

18. The method of claim 1 wherein the risk management intervention program comprises:
distributing interventions to multiple end users, wherein the multiple end users are selected from the group consisting of physicians, pharmacists, health care providers, caregivers and patients.

19. The method of claim 1 wherein the risk management intervention program comprises:
coordinating care among multiple end users, wherein the multiple end users are selected from the group consisting of physicians, pharmacists, health care providers, caregivers and patients.

20. The method of claim 1 wherein the risk management intervention program comprises:
tailoring said risk management intervention program to local medical practice standards and needs including, but not limited to the delegation of primary responsibility for the program from physician to support staff.

21. The method of claim 1 wherein the risk management intervention program comprises:
designing interventions that are effective in transferring medical knowledge and reinforcing knowledge retention.

22. The method of claim 1 wherein the risk management intervention program comprises:
utilizing one or more of adult learning principles, enablers, personal application, multiple media, repetitive messaging, self assessments, feedback, incentives and consequence messages.

23. The method of claim 1 wherein the risk management intervention program comprises:
developing a risk communication curriculum to communicate risk to an end user, wherein the end user is selected from the group consisting of physicians, pharmacists, health care providers, caregivers and patients.

24. The method of claim 1 wherein the risk management intervention program comprises:
transferring know-how, insights, techniques, methods, and processes from more experienced physicians and support staff to less experienced physicians and support staff.

25. The method of claim 1 wherein the risk management intervention program comprises:
utilizing existing interventions and tools developed by one or more of clinicians, peer to peer forums, clinical consultations and preceptorships.

26. The method of claim 1 wherein the risk management intervention program comprises:
implementing human behavior changing interventions.

27. The method of claim 1 wherein the risk management intervention program comprises:
utilizing disease management approaches, principles, methods, techniques and tools to change end user behavior.

28. The method of claim 1 wherein the risk management intervention program comprises:
integrating risk messages into promotional materials of the pharmaceutical product.

29. The method of claim 1 wherein the risk management intervention program comprises:
utilizing a professional support network for the collection and management of data associated with the adverse events.

30. The method of claim 29 wherein said data comprises:
occurrences of the adverse events.

31. The method of claim 1 wherein the risk management program comprises:
educational resources for delivering information regarding prescribing, dispensing, and use of the pharmaceutical product.

32. The method of claim 31 wherein the educational resources comprises:
identification of control measures for the pharmaceutical product.

33. The method of claim 31 wherein the educational resources comprises:
classes to instruct and end user on said control measures.

34. The method of claim 31 wherein said educational resources are available by electronic, written, audio, or video communication.

35. The method of claim 1 wherein the risk management intervention program comprises:
implementing distribution controls wherein said distribution controls manage the availability of the pharmaceutical product.

36. The method of claim 35 wherein the distribution controls comprise:
limiting availability of the pharmaceutical product to a single source.

37. The method of claim 35 wherein the distribution controls comprise:
limiting availability of the pharmaceutical product to authorized pharmacies.

38. The method of claim 35 wherein the distribution controls comprise:
requiring a pharmacist to be certified to dispense the pharmaceutical product.

39. The method of claim 35 wherein the distribution controls comprise:
limiting physician prescribing rights.

40. The method of claim 39 wherein limiting physician prescribing comprises:
limiting the number of refills per prescription, limiting the expiration date of a prescription, and/or limiting the form of a prescription.

41. The method of claim 1 wherein the risk management intervention program comprises:
mandating periodic or intermittent tests for the existence of contraindications for the pharmaceutical product.

42. The method of claim 41 wherein said contraindications comprise pregnancy.

43. A method for creating educational materials for use in mitigating the risks of a pharmaceutical product comprising:
compiling a database of educational components wherein said components are useful in providing information regarding the risks of a pharmaceutical product or procedures to mitigate said risks;
selecting a plurality of said components from said database to create an educational tool kit wherein the components are selected based on the expected effectiveness of said component in managing the identified risks of the pharmaceutical product;
tailoring interventions to assure implementation by other end users;
designing a dissemination plan for distributing the educational materials to a target audience;
implementing the dissemination plan through forums and venues, such as learning labs, virtual learning forums, and preceptorships;
evaluating the expected impact of the educational tool kit on the target audience; and
adding, deleting, or modifying the selected components to achieve a desired expected impact.

44. The method of claim 43 further comprising:
integrating a measurement of actual effectiveness of said components of said educational tool kit into said database.

45. The method of claim 43 wherein said database is compiled from existing risk management programs.

46. The method of claim 43 wherein the target audience comprises a patient being administered the pharmaceutical product.

47. The method of claim 43 wherein the target audience comprises a patient's caregiver or family member.

48. The method of claim 43 wherein the target audience comprises a physician prescribing the pharmaceutical product.

49. The method of claim 43 wherein the target audience comprises a physician's clinical or office staff.

50. The method of claim 43 wherein the target audience comprises a pharmacist dispensing the pharmaceutical product.

51. A system comprising:
a logic configured to identify, characterize and rank adverse events caused by using a pharmaceutical product;
identify a medication use process associated with the pharmaceutical product;
identify potential failure modes of the medication use process;
quantify the potential effect of said failure modes to create a pharmaceutical hazard score, wherein said hazard score considers the severity and frequency of occurrence of the effects of said failure modes;
conduct a logical hazard assessment of said failure modes found to have a high hazard score to evaluate the need to mitigate the effect of said failure modes; and
design a risk management program to manage said adverse events.

52. A system comprising:
a processor;
a memory operably connected to the processor, where the processor can access the memory;
a logic operably connected to the processor, where the logic is configured to:
identify, characterize and rank adverse events caused by using a pharmaceutical product;
identify a medication use process for the pharmaceutical product;
identify potential failure modes of the medication use process;
quantify the potential effect of said failure modes to create a pharmaceutical hazard score, wherein said hazard score considers the severity and frequency of occurrence of the effects of said failure modes;
conduct a logical hazard assessment of said failure modes found to have a high hazard score to evaluate the need to mitigate the effect of said failure modes; and
design a risk management program to manage said adverse events wherein said risk management program comprises control measures to reduce the incidence and consequences of said failure modes.

53. A computer-readable medium storing processor executable instructions operable to perform a method, the method comprising:
identifying adverse events caused by using a pharmaceutical product;
identifying a medication use process for the pharmaceutical product;
identifying potential failure modes of the medication use process;
quantifying the potential effect of said failure modes to create a pharmaceutical hazard score, wherein said hazard score considers the severity and frequency of occurrence of the effects of said failure modes;
conducting a logical hazard assessment of said failure modes found to have a high hazard score to evaluate the need to mitigate the effect of said failure modes; and
designing a risk management program to manage said adverse events wherein said risk management program comprises control measures to reduce the incidence and consequences of said failure modes.

54. A system comprising:
a means for identifying adverse events caused by using a pharmaceutical product;
a means for identifying a medication use process for the pharmaceutical product;
a means for identifying potential failure modes of the medication use process;
a means for quantifying the potential effect of said failure modes to create a pharmaceutical hazard score, wherein said hazard score considers the severity and frequency of occurrence of the effects of said failure modes;
a means for conducting a logical hazard assessment of said failure modes found to have a high hazard score to evaluate the need to mitigate the effect of said failure modes; and
a means for designing a risk management program to manage said adverse events wherein said risk management program comprises control measures to reduce the incidence and consequences of said failure modes.
a means for measuring, evaluating and reporting the effectiveness of a risk management program.

55. A pharmaceutical product risk assessment and management kit comprising:
one or more intervention components, wherein said one or more intervention components are selected for inclusion in said pharmaceutical product risk assessment and management kit by a pharmaceutical product risk assessment and management method.

56. A pharmaceutical product risk assessment and management kit of claim 55, wherein said kit is selected from the group consisting of a patient kit, a physician kit and a pharmacist kit.

57. A pharmaceutical product hazard scoring chart comprising:
a first column of a pharmaceutical severity scale; and
a second column of a pharmaceutical frequency of occurrence scale;
wherein a pharmaceutical hazard score is the product of said first column and said second column.
